(19) United States
(12) Patent Application Publication
(10) Pub. No.: US 2001/0047185 A1
(43) Pub. Date: Nov. 29, 2001

(54) Radioactivatable composition and implantable medical devices formed therefrom

(76) Inventor: Stanley Satz, Surfside, FL (US)

Correspondence Address: CLIFFORD G. FRAYNE, Suite 7A, 136 Drum Point Road, Brick, NJ, 08723, US

(21) Appl. No.: 09/817,307

(22) Filed: Mar. 26, 2001

Related U.S. Application Data
(63) Continuation-in-part of application No. 09/138,594, filed on Aug. 22, 1998.

(51) Int. Cl. A61M 29/00; A61F 2/06

(52) U.S. Cl. 606/198; 623/1.15; 623/1.43; 424/426; 604/104

(57) ABSTRACT

Disclosed are radioactivatable compositions, preferably metal alloy compositions containing a metal having shape memory characteristics, and at least one radioactibatable isotope comprising a lanthanide series element or mixtures of lanthanide series of elements or other suitable isotope. The radioactivatable isotope is present in sufficient concentration (relative to other components of the composition) to deliver an effective radiation dose to a target tissue to achieve a specified therapeutic objective. One of the more advantageous and useful applications for this composition is the formation of medical devices for the treatment of coronary artery disease and the abatement of proliferation of cancer cells. In one of the embodiments of this invention, a radioactivatable isotope is incorporated, by isotopic beneficiated combination, with a matrix material such as nickel/titanium alloy (e.g. Nitinol metal alloys), or by isotopic beneficiated combination with a biodegradable organic naturally occurring or synthetic polymer so as to form a solid solution; and, the resultant alloy or solid solution, therafter, formed into a stent, or other suitable form, for selective, targeted delivery of therapeutic and effective amounts of low dosage radiation (e.g. beta particles) to a specific site or tissue within the body.

2. Related Applications

This application is a continuation-in-part of U.S. Ser. No. 09/138,594 filed Aug. 22, 1998, which was a continuation-in-part of U.S. Ser. No. 09/038,560, filed Mar. 11, 1998.

### BACKGROUND OF THE INVENTION

1. Field of the Invention

This invention relates to a composition of matter and useful articles formed from such compositions and more particularly to a radioactivatable composition and implantable medical device formed therefrom.

2. Description of the Prior Art

The target specific delivery of drugs and medical devices for the abatement and prevention disease is beginning to come of age, although not without certain limitations and problems associated with both administration and delivery. Among the medical procedures that currently use such a target specific approach is treatment of coronary artery disease by drug mediated intervention, balloon angioplasty and, more recently, intra-arterial radiation therapy. Restenosis, in which occluded coronary arteries reclose within six months after being dilated by balloon angioplasty, occurs in forty percent (40%) or more of patients, usually within six months or less, and continues to remain a serious limitation to long term success of balloon angioplasty.

One targeted radiation therapy in cardiovascular applications is the use of a radioactive liquid filled balloon (containing, for example rhenium-186) for the treatment of restenosis. The preparation and filling of a balloon with a radioactive solution such as rhenium-188 is complicated by the fact that several steps are involved in the preparation of such device, and the ever present potential for bursting. The resultant balloon is subject to many of the same shortcomings and frailties of balloons currently in use in angioplasty procedures.

Another targeted radiation therapy of cardiovascular applications is the use of stents with concomitant use of radioactive procedures.

It is evident that there is and continues to remain a number of unresolved problems associated with the use and delivery of therapeutic dosages of radiation to target tissues for the treatment and abatement of disease states.

The prior art is deficient in one or more material aspects of targeted delivery of radiation therapy with an unfulfilled need for therapeutic radioactive device having both efficacy and safety for use in a clinical setting.

### OBJECTS OF THE INVENTION

An object of the present invention is to provide a radioactivatable composition in the fabrication of medical devices.

A further object of the present invention is to provide a radioactivatable composition suitable for fabrication of biomedical devices, including implantable intra-arterial stents.

Yet another object of the present invention is the use of a radioactivatable compositions for medical devices for delivery of combinations of radiation and companion therapies, to provide both immediate and extended treatment of targeted tissue.

### SUMMARY OF THE INVENTION

These and other objects of the present invention are achieved by a radioactivatable composition having both physical and nuclear properties suitable for fabrication of biocompatible medical devices and the use thereof in the targeted delivery of radiation therapy in the treatment of coronary artery disease, specifically, arterostenosis, restenosis (following balloon angioplasty) and instent restenosis.

### DESCRIPTION OF THE INVENTION

In the adaptation of memory metal alloys, the relative stoichiometry of the alloy components, as is the processing history, is of critical importance to control of the physical properties of the resultant product. Accordingly, the efficacious modification of a matrix material, such as an alloy, by the inclusion of a radioactivatable isotope, is unpredictable because such properties are recognized as dependent upon the precise proportions of the major components of the matrix material and, thus, must be undertaken with extreme care. Moreover, the nuclear properties of the isotope (e.g. stability) are also, to a degree, dependent upon their interaction with the other (major) components of the composition, under the processing conditions required for their combination, and can, thus, also produce unexpected and unpredictable results.

In each instance, the resultant device and/or item is thereafter activated by exposure in a nuclear reactor by N-gamma or other reaction from a neutron source such as a nuclear reactor, or by a proton beam in an accelerator or a cyclotron, so as to energize the radioactivatable substance within the composition prior to use, and thereby cause short range emission of low level radiation (preferable beta particles) from the device and/or item, over a finite period (half life) depending upon the specific radioactivatable substance of choice.

The radioactivatable substance is selected from the isotopic form of the lanthanide series of elements in the periodic table of elements, and most preferably from a group consisting essentially of lutetium-177, samarium-153, cerium-137, 141 or 143, terbium-161, holmium-166, erbium-166 or 172, thulium-172, ytterbium-169, yttrium-90, actinium-225, astatine-211, cerium-137, dysprosium-165, erbium-169, gadolinium-148, 159, holmium-166, iodine-124, titanium-45, rhodium-105, palladium-103, rhenium-186, 188, scandium-47, samarium-153, strontium-89, thulium-172, vanadium-48, ytterbium-169, yttrium-90, silver-111, and mixtures thereof. Of the aforementioned lanthanides, lutetium-177 is particularly preferred for its known chemical versatility and therapeutic value.

One or more radioactivatable isotopes are combined in the appropriate proportions in uniform dispersion with a biocompatible metal or a biocompatible polymer (hereinafter also “matrix material” or “matrix”) and the resultant mixture processed by mechanical means, such as melt mixing or twin screw extrusion so as to form the composition. The composition, in the case of the metal alloy, is typically vacuum arc melted and thereafter progressively cooled (annealed) to form a product that can be fabricated into useful shapes and articles of manufacture. Similarly, the composition, in the case of a polymer, can be melt mixed, extruded or solution blended and thereafter can be recovered as compound, extruded, solvent cast, or drawn through a spinneret as a fiber, from which useful shapes and articles can be manufactured. The biocompatible polymer can typically comprise any readily processable organic and/or organometallic polymerizable substance having the requisite physical and processing characteristics to accept the isotope, at the appropriate concentration, and yet reset the activation energy required to energize isotope, incident to its use. These materials typically include the same polymeric materials currently available and in use in the medical devices in the catheterization laboratory, specifically, the polyurethanes, polyamides, polyvinyl chloride, methylmethacrylate and their various combinations (e.g. graft and block copolymers).

The resultant product is virtually free from leaching or flaking (as is the case of medical devices-coated with radioactive phosphorus-32), and exhibit precise control of the radiation dose, (e.g. low radiation dose, and shallow tissue penetration) and, thus, provide for substantial improvement in the means of therapeutic delivery of radiation to mannalian tissue. Moreover, when the medical device is a stent, it can be prepared several days or weeks in advance by precalibration (producing a higher level of radiation that decays to the desired delivered doses) and shipped and stored until needed for use. At the time of receipt and/or prior to implantation by the hospital, the radioactive stent should be and remain active for at least 24 hours up to about 10 days.

The radioactivatable composition (and the medical devices formed from these materials), retains its native desirable physical and chemical properties of the metal and polymer matrix material, respectively; and, thus, these metal and polymer compositions are preferably selected from known metals (included alloys) and polymers that are known to be useful in the fabrication of medical products and devices.

The radionuclides that can be used in the present invention, will be alpha, beta or Auger emitters of therapeutic value and with a half life sufficiently long to make the activation, preparation and shipment of the radioactivatable devices practical. Therefore, radionuclides with a half-life of at least 24 hours are preferred. Radioactive elements, such as calcium, utilizing present delivery systems, are potentially undesirable because they chemically react when in direct contact with blood. Likewise, radionuclides that require long irradiation times are also inexpedient and can give rise to undesirable long lived or gamma emitting radioisotopes that result from impurities within the nickel, titanium or chromium matrix. Moreover, to the extent a relatively large quantity of the enriched stable isotope is required (in excess of the amount that can be effectively “dissolved” within the matrix without phase separation and/or material alteration of processing conditions), the materials balance of the matrix will be adversely affected resulting in an unacceptable temperature transition temperature and, thus, the resulting intra-arterial deployment of the device being affected. Moreover, if the natural or enriched stable isotope is incompatible with the matrix material in terms of, say melt temperature, it obviously cannot be used. Similarly, enriched or natural stable isotopes that give rise to long lived radionuclides are also generally considered of marginal value for this critical application. Accordingly, the radioisotopes of choice possess the requisite desirable characteristics of short nuclear reactor or cyclotron activation time, small amount of radioactivatable stable isotope required within the carrier matrix, a beta emitter with preferably a small gamma emission for imaging purposes, compatibility with mammalian tissue and blood, desirable half life, e.g. more than 24 hours but less than 60 days.

The radioactivatable composition comprises a metal or metal alloy of nickel and titanium containing a uniform dispersion of from about 0.01 to about 10 weight percent of a radioactivatable isotope from the lanthanide series of elements. The relative weight ratio of nickel and titanium in the composition is preferable the same as typically used in the so-called “Nitinol” or “memory metal” family of alloys prepared from these materials. In the context of this invention, the alloy is proportioned and processed to have memory effects at or slightly below the temperature of the environment of intended use, e.g. memory metal effects @ 33° C. for use in intraluminal environment of human body. Thus, the shape memory metal alloys, preferably a ternary alloy, are produced so that when activated, both emit radiation and yet retain their otherwise native and desirable combination of physical and therapeutic properties.

The compositions are preferably formed from superelastic materials (e.g. nickel/titanium alloy); and, are intended for the fabrication of radioactive wire, tube or mesh and, as such, are especially suited for various designs of medical implant used in the treatment of cardiovascular or oncological disease. The method of manufacture of the compositions of this invention, thus, involves combining radioactivatable additions of a stable or enriched isotope and a nickel/titanium alloy to a near stoichiometric nickel titanium or nickel chromium alloy, so as to alter the atomic percent ratio of the Ti and Al or the Ni and Cr to what has been found to be an effective alloy. In one embodiment, a stable isotope such as lutetium-176, or other inclusion which may be optionally coupled with additions of other radioactivatable dopants or combination of dopants selected from a group consisting of natural or enriched stable isotopes or combination of stable isotopes thereof, are made in approximate concentrations of between 0.0025 and 10 atomic percent.

A preferred composition for the foregoing superelastic composition can be approximated by the following expression wherein the proportion/ratio of the components of the matrix (e.g. alloy) can be adjusted relative to the amount of isotope that is present therein:

Ti--i Ni (48--51)Lu(0.0025--10)

Ni--i Cr (48--51)Lu(0.0025--10)

Ti(x)Ni(y)Me(z)−(x+y+z)

in which Me is at least one natural or enriched stable isotope that when irradiated gives rise to a radioactive isotope, when present in approximate concentrations of between 0.0025 and 10 atomic percent.

The “Me” is selected based upon both practical criteria and functional constraints dictated by its environment of intended use. For example, it is generally preferable to select a radioactivatable isotope that requires relatively little activation energy to form the corresponding radioactive analogue having a half-life time within the preferred parameters at least 24 hours and less than 10 days. Moreover, the nuclear response of the preferred radioactivatable isotope to low activation energy generally favors the formation a single isotope having primarily beta particle emission without giving rise to other isotopes whose nuclear properties emit gamma radiation or that have extended life times. Lutetium is the model for the preferred radioactivatable isotope. More specifically, lutetium is characterized by low energy beta emissions, short half life and due to a very wide cross section in Barns, ease of activation at low power (neutron flux rate) in a nuclear reactor. The incorporation of this enriched stable isotope within a metal or shape memory alloy, while at very low percentage, does not have an appreciate effect upon shape memory characteristics, and is yet sufficient for activation thereof in a nuclear reactor. Although interstitial Lutetium atoms have a larger size (Z=71) and could theoretically alter the lattice structure Nitinol alloys, empirical data appear to indicate essentially no substantial change in the alloys modules of elasticity and the dispersivity at the optimum Lutetium concentration (0.05-0.1), thus retaining the original Nitinol alloys properties and stents fabricated from this novel ternary alloy. At the concentration contemplated herein of from 0.01 to about 10 weight percent the lutetium doped nickel/titanium alloys form a meltable, castable, weldable, bondable, magnetic or non-magnetic cohesive composition that can be activated and made radioactive, whilst resistant to corrosion or reactivity in blood over a wide range of acid strengths.

With its wide cross-section, lutetium results in rapid activation in a low-power nuclear reactor with short irradiation time as a low flux rate. By being able to use a short irradiation time at relatively low flux rates, production costs are reduced. Furthermore, when utilizing a natural or highly enriched stable isotopic form of lutetium-176, the formation of undesirable long lived isotopes, such as high energy beta emitters or deeply penetrating gamma emitters is avoided. The advantages of a lutetium-176 doped composition are, thus, indeed both significant and unexpected. Since only less than 10% of an enriched stable isotope is required as a part of the device, (and in the case of some isotopes such as lutetium-176, preferably as low as 0.10 percent), the neutron penalty is low, the irradiation time in the reactor may be brief, the shortened irradiation time reduces the possibility of giving rise to undesirable long lived radioisotopes which can result from inorganic impurities, the reactor core size may be minimal, the irradiation flux requirement can be reduced, and the nuclear waste disposal volumes would be small. Further advantage occurs by the addition of a quantity of one or more of an isotopically enriched elements. When exposed to radiation in a reactor, such a material, preformed or post formed, produces only short half-life radioisotopes. Another advantage of this radioactive material is reduced nuclear waste disposal problems as a result of much shorter isolation time and decay requirements. As beta emitting radioisotopes travel only a short distance, radionuclides of this type are most desirable, in particular where there is only a weak gamma facilitating device visualization and calibration. In another preferred embodiment, the maximum soft tissue penetration of short lived lutetium-177 (6.67 day half life) is 0.15 millimeters.

Only short reactor irradiation time is, thus, required for the preferred Lutetium doped compositions of this invention to achieved desired levels of radioactivity, preferably between 20 microcuries and 50 millicuries, when activating isotopically enriched or natural lutetium. On the other hand, if nickel titanium or chrome nickel is activated to yield, say, vanadium-22, long lived radioactive impurities and high energy gamma emitters have been known to arise. Unlike most other radioisotopes, such as yttrium-90 produced from yttrium-89 wire, much higher specific activities can be achieved utilizing lutetium-177 without giving rise to undesirable radioisotopes.

The present invention provides a unique range of radioactive alloys for the radioactivatable compositions, wherein there is provided either a single enriched stable isotope or combination of enriched stable isotope or isotopes, including tellurium, germanium, iodine, monoisotopic yttrium or other element, which may be a natural or isotopically enriched form of an element. For example, an alloy may optionally be doped with a combination of beneficiated stable isotopes, including preferably lutetium-176, samarium-152, strontium-88, yttrium, or other natural or enriched stable isotopes. Depending upon the relative concentration of isotopes and the environmental constraints imposed by the anticipated use, the composition shall only require relatively short nuclear reactor irradiation time at low neutron flux rates to achieve desired levels of radioactivity, preferably between 20 microcuries to 50 millicuries, when activating a unique alloy containing isotopically enriched or natural lutetium.

Because of the impurities typically found in metal alloys, organic polymer based compositions may have certain advantages; and, to the extent that “memory” can be engineered into such polymeric materials, would be the system of choice. Typically, polymer composition of this invention can be prepared by an admixture of a biocompatible resin and an enriched stable isotope, or combination of isotopes, preferably lutetium-176 so as to yield radioactive lutetium-177 (6.71 day half life), which is produced by neutron capture irradiation from isotopically enriched (70-75%) lutetium-176. As above noted, and once again emphasized, radioactive lutetium-177 is principally a beta emitter, most energy deposited only penetrates a few millimeters into contiguous tissue, ˜0.15 mm (78.2% at 497.3 keV, 12/2% at 176 keV and 9.5% at 384.3 keV); and, exhibits a weak gamma (11% at 208.4 keV and 6.5% at 112.9). Radioactive lutetium-177 decays to metastable hafnium-177. Further, the incorporation into the polymer of lutetium-177 takes advantage of the inherent safety advantages of a short lived, short range, low-dose beta radiation emitter by incorporating the polymer-encapsulable lutetium-177. this isotope has a weak but measurable gamma emission, so as to overcome the problem of dose calibration.

In another embodiment of the present invention, en enriched stable isotope, preferably lutetium, (which typically exhibits spontaneous infiltration properties under a given set of processing conditions) can be induced to infiltrate a metal or alloy when combined or contacted with a matrix metal having either a physical form or affinity for the isotope so as to be receptive to spontaneous infiltration properties of the Lutetium. It is known, for example, that when an infiltration enhancer and/or an infiltration enhancer precursor and/or an infiltrating atmosphere are in communication with a filler material or a preform, at least at some point during the process, and a metal which, under the process conditions, ordinarily would not exhibit spontaneous infiltration, is combined with (e.g. mixed with and/or exposed to) a matrix which does exhibit spontaneous infiltration behavior under the same processing conditions, the combination of metals will spontaneously infiltrate the filler material or preform.

The materials and processes of the present invention are especially useful for the preparation of radioactive shape memory alloys that transition at or near body temperature and relates to a process for preparing and forming novel, medically useful radioactively beneficiated compositions for the forming of biocompatable implantable stents therefrom. In use, the devices provide localized, sustained release of a uniform, short-lived, low-level radiation dose. Unlike gamma emitters, the radiation is confined so that very limited radiation is delivered to nearby healthy tissue. Thus, the radioactive stents of this invention provide a novel, clinically practical approach to the prevention of restenosis after angioplasty and the treatment of certain cancers. Lutetium-177 further provides radioopacity and may also be imaged using various nuclear medicine modalities including single photon emission computed tomography, gamma camera, scinitigraphy, PET, or alternatively, autoradiography, fluoroscopy or X-ray.

The radioactivatable composition can be converted into a tube, a wire or mesh, and may be braided, woven, knitted, or wound together, or laminated, wherein thee enriched stable isotope is uniformly dispersed and incorporated throughout the radiation delivery component of the medical device (e.g. stent). Where the medical device is a stent, it is contemplated that such device can be utilized intra-arterially or interstitially in its non-radioactive state. The radioactivatable compositions are particularly well suited for the preparation of radioactivatable stents and radioactive meshes that may be easily handled for use in the treatment of vascular disease, cancer, benign prostatic hyperplasia and other diseases. The device fabricated from the composition of this invention may be activated by irradiation/neutron bombardment in a nuclear reactor, or by proton or electron beam in a cyclotron or accelerator, resulting in a radioactive stent.

The radionuclide selection criteria, as above described herein, results in a radioactive stent that can be stored indefinitely and readily disposed of with practical consideration being given to the half life of the radionuclide. This intended period of storage is practically limited by the half life of the radioisotope. In the case of Lu-177, for example, the desired period of storage would range from 0 days to about 20 days. Thus, the radioactive stent could be shipped to end users of the product and could be implanted with very little additional preparation time or effort than a conventional non-radioactive stent.

The radioactivatable stent can include or be coated with other components (hereinafter “companion substances”), if desired. Useful therapeutic compounds that can be associated with the stent and, thus, delivered at a controlled release rate, include anti-proliferative drugs such as GP IIb-IIIa platelet inhibitors, benign prostatic hyperplasia inhibitors, chemical stabilizers such as ascorbic acid, gentisic acid and for the diffusion of anti-telomerase compounds and anti-neoplastic drugs including cytarabine, doxorubicln vincristine and cisplatin. A radiolytically stable biocompatible radioactive polymeric gel for use as an arterial or body passageway paving material or coating is also contemplated for use with the products of this invention. These companion substances, together with the radionuclide, may be incorporated within a biosorbable polymer matrix such as a hydrogel, a lactide, polyglycolic acid, a poly(beta-hydroxybutyric acid), poly-DL lactic acid, containing a radioactivatable substance for combination or adjuvant therapy. Thus, a stent made of these materials, or coated with these substances, would provide combination therapy by both emitting radiation and delivery of a therapeutic substance in-situ. It is emphasized that the co-application of such therapy is not simply accretive, but rather enables the more efficacious treatment of the physiologic condition or disease state by permitting an initial radiation treatment to shock or arrest the undesirable physiological processes, and thereafter delivery of a sustaining therapy (possible at a lower dosage) to the site specific target for treatment.

Biodegradable radioactivatable stents are principally comprised of any one of the following polymers or copolymers compounds or hydrogels: lactides, glycosides, caprolactones, oxyalkanes, polyurethanes, and ultra high molecular weight polyethylene. These compounds or hydrogels can contain a radiation emitter such as lutetium-177, samarium-153, cerium-137, 141 or 143, terbium-161, holmium-166, erbium-166 or 172, thulium-172, ytterbium-169, yttrium-90, actinium-225, astatine-211, cerium-137, dysprosium-165, erbium-169, gadolinium-148, 159, holmium-166, iodine-124, titanium-45, rhodium-105, palladium-103, rhenium-186, 188, scandium-47, samarium-153, strontium-89, thulium-172, vanadium-48, ytterbium-169, yttrium-90, silver-111; or a combination thereof or other radioisotope with a half life of less than two months, preferably one that principally emits a short lived alpha, preferably a beta emitter or an Auger electron.

The biodegradable radioactivatable stent safely degrades within the bloodstream over a period of weeks or months. The radioactivatable biodegradable stent undergoes progressive erosion and/or decomposition into harmless materials and the radioactive component of the short lived radioisotope will have decayed to ultralow, safe levels and thus overcomes mechanical limitations and permanency associated with metallic stents. These devices, thus, provide a “scaffold” for remodeling the vessel as well as a pharmacokinetically acceptable vehicle for sustained local drug delivery, and as such can provide an alternative to prevent restenosis and acute closure post PTCA.

An implantable deformable polymeric stent, made from the radioactivatable polymers of exhibit enhanced mechanical and processing properties in response to polymer modification by activation, and thus enable the incorporation of a organometallic (such as an organotitanate, an organozirconate or an organovandate) additive as a processing aid for enhanced linking of the organic and inorganic radioactivatable component, while providing uniform and selective radiation delivery to the target tissue.

Biodegradable terpolymers or hydrogels containing a short lived radioisotope exhibit controlled bioerodability and bioresorption degrading over time into harmless materials. These polymers, terpolymers, homopolymers, copolymers, oligomers, or a blend thereof such as a poly (DL-lactide-co-glycolide) and selected monomers, oligomers or terpolymers, may be used to form a radioactive stent providing sustained, site specific adjunctive drug delivery. The group of radioactive polymers includes selected lactides and shape memory plastics. Other radioactive, bioabsorble polymers suitable for this purpose include lactides polyglycolic acid, polyorthoesters, (utilized for the sustained release of contraceptive steroids), glycosides, polyanhydrides, phosphazines, caprolactones, oxyalkanes, trimethlene carbonate, paradioxanone, polyacryl starches, triethyleneglycol monomethylacrylate, hydrogels, polyurethanes, and other potentially radioactive terpolymers which undergo decomposition bioerodable and bioabsorbable terpolymers including polyglycolic acid, poly(2-hydroxyethyl methacrylate), poly L-lactic acid, poly (e. caprolactam), poly (DL-lactide-co-glycolide) high molecular weight poly-L-lactic acid poly L-lactide, polyglycolic/poly-L-Lactic acid, polyglactin, polydioxanone, polyglyconate, e-caprolactone, polyhydroxybutyrate valarate, covalently immobilized poly(2-hydroethylmethacrylate)-gelatine composite polymer, polyethylene terephthalate (PET polyanhydride), ethyl terminated oligomers of lactic acid, difunctional polyurethane, and radioactive copolymers of any combination of the aforementioned materials such as 50/50 (poly)D,L-lactide-co-glycoside.

The compositions of the present invention can be converted into a radioactive tube, strand, fiber, thread, mesh, film, coil or polymer coated wire and may be braided, woven, knitted, crocheted, wound, (or any combination of the aforementioned procedures, preferably knitted, braided, and woven) multilayered, molded, extruded, cast, welded, bonded, glued, high frequency or ultrasonic welded or heat sealed into a predetermined shape constituting a stent, in which a natural or enriched stable isotope is uniformly dispersed in particle form and incorporated throughout the stent material. A compressed radioactivatable stent can be prepared by knitting, weaving, braiding or a combined method thereof of a biostable or biodegradable polymeric fiber, filament or a combination of a polymer fiber or filament and a wire.

A stent may be coated with a radioactive/radioactivatable hydrogel which may contain a minimally platelet activating, anti-thrombolytic or anti-proliferative agent as a platform for the delivery of a drug to further inhibit the proliferation of neointima. The coating of an intravascular radioactive stent with a hydrogel is a means of precisely targeted high dose drug delivery with a sustained biological half life. Therapeutic drugs that may be delivered at a controlled release rate include anti-proliferative drugs such as GP IIb-IIIa platelet inhibitors, anti-neoplastics, benign prostatic hyperplasia inhibitors, chemical stabilizers such as ascorbic acid, gentisic acid and fo the diffusion of anti-telomerase compounds and anti-neoplastic drugs including cytarabine, doxorubicin vincristine and cisplatin. A radiolytically stable biocompatible radioactive polymeric gel for use as an arterial or body passageway paving material or coating is also claimed.

In a preferred embodiment the stent may also be coated with the aforementioned gel which contains a minimally platelet activating, anti-thrombolytic or anti-proliferative agent such as a nitric oxide donor, or may be the platform for the delivery of a drug to further inhibit the proliferation of neointima. Thus, a radioactive stent may be coated with heparin, coumadin, dexamethasone, ticoplidine, nitric oxide, other pharmaceutical agent or a biologically active substance so as to enable the delayed release of a pharmaceutical or a recombinant compound and to further reduce the risk of thromboses in combination with intraarterial brachytherapy. Alternatively, the polymer may contain any of the aforementioned agents by incorporating mixing said agent into the polymer prior to the production of the finished shape.

Organometallic chelators can be used in combination with the isotopes to link various other substances to such isotopes to provided combination therapies. Typically this involved obtaining a polymer with improved dispersion and cohesive bonding of additive components comprised of the aforementioned applicable polymers (including other polymers that may be substituted are polyanhydride polymer such as polyethylene terephthalate (PET), polyurethanes, polyethylene oxide, ultra high molecular weight polyethylene, polynorbornene, or a copolymer such as fluorine-acryl-styrene-urethane-silicone, 2-[2′-iodobenzoyl]-ethyl methylacrylate and hydrogels containing azoaromatic moieties), and the use of titanium, zirconium, vanadium or iodine organometallic coupling and processing agents as an aqueous solution or a powder such as an organotitanate to enable combining of different biodegradable polymers with a radioisotope. The aforementioned chelate, or mixtures thereof, may be used to link radioisotopes, such as lutetium, samarium or other activatable isotope and/or substance or drug to a range of polymers, so as to cross-link and enhance dispersive and siccative properties or to improve the adhesion between the organic and inorganic components, improving flowability and reducing voids in precursors. The cross-linking reaction modifies the inorganic surface by forming a monomolecular organic complex layer due to a cross-linking reaction between the organotitanate, or other organometal, and the polymer causing complete dispersion of the radioactive particles or fibers. The organometallic may be used to surface treat a polytetrafluorethylene to improve the binding characteristics to drug compounds.

The following examples are illustrative of the present invention.

### EXAMPLE 1

A radioactivatable ternary alloy charge comprising 53.1 weight percent nickel, 0.1 weight percent lutetium, and 44.8 weight percent titanium weighing 50 grams is placed in a crucible. Prior to melting, deoxidization is performed by striking a movable arc onto a zirconium getter source. The alloy charge is vacuum arc melted and flipped three times at 1,750° C. to form a button. The resulting alloy is cast in a second copper crucible at the or about the same temperature into a ⅝ inch diameter rod under an inert atmosphere.

### EXAMPLE 2

The resulting 0.480″×2.75″ rough rode of Example 1 is machined on a lathe to achieve a smooth, clean surface and is inserted into a stainless steel tube. The ends of the stainless steel tube are welded closed. The assembly is hot swaged using progressive steel dies at 500° C. so as to convert the sample to an ⅛″ rod whereupon the stainless steel is peeled off the Ni—Ti—Lu sample. In order to render the rod and the resulting wire ductile, it was necessary to heat the wire to about 500° C. The final annealing temperature causes a shift in the transition temperature for the radioactivatable alloy of this given composition. The rod is subsequently hot drawn into wire using twenty progressive tungsten carbide and diamond dies, annealing for 30 minutes after each pass. The wire is reduced in diameter to 0.015 inch and carrying lengths were annealed at temperatures ranging from 450° C. to 600° C.

### EXAMPLE 3

the wire formed according to Example 2 is thereafter annealed. Annealing of the radioactivatable alloy is done at a high temperature well above the Af. On cooling the material stays austenite until the Ms temperature is reached. Further cooling causes the austenite state to transform to martensite with the transformation being complete at Mf. On heating the martensite is stable until the As is reached. Further heating causes the martensite state to transform with the transformation being complete at the Af. If the heating or cooling of the radioactivatable alloy is stopped before the transformation is complete the amount of each phase present will be stable. Between the Ms and As the radioactivatable alloy can exist in either phase or combination of phases depending upon the thermal treatment history. Thus the ingot temperatures were: Mf=2° C., Ms=27° C., As=46° C. and Af=75° C.

For the production of radioactivatable shape memory Nitinol wire, the wire is preferably 100% austenitic (where it is to be formed into a knitted or braided tube stent). Thus, the wire is heated above the Af and was kept above the Ms until the tubular shape was produced. The device is thereafter cooled below the Mf and kept below the Af for forming.

As the radioactive stent heats above the As to the Af, it will take the original knitted or braided shape. The Af is near mammalian body temperature, (37° C.). Ninety to ninety-five percent (90-95%) transformation may be considered acceptable. However, the As should be as high as possible before insertion is completed with about a 5-10% transformation occurring before insertion is completed. Transformation may be restrained by sheathing. The transformation temperature, (Af), may be adjusted by adjusting the alloying elements but the Af-As tends to be fixed.

### EXAMPLE 4

Radioactivatable NiTiLu wire of 0.019″ diameter, of Example 2, annealed at 520° C., completed its memory response at 36.1° C. in water (as measured with a thermocouple). Thus, as the radioactivatable alloy is warmed by body heat, (which is above the temperature transition range), it expands and regains its permanent shape; and, in the case of a radioactive implantable medical device, such as a stent, displaces surrounding tissue in the process.

### EXAMPLE 5

A 0.0058″ (260 mm length) wire sample of the radioactivatable alloy of Example 1 (53.1 weight percent nickel 0.1 weight percent lutetium, and 44.8 weight percent titanium) weighing 27.8 mg.—containing approximately 0.0278 mg. of lutetium—is placed in a quartz glass protected with aluminum foil. The tube is placed into an aluminum capsule holder, pressure sealed using an inert gas and welded shut. The capsule is inserted into a reactor channel positioned by hydraulic means and activated by neutron activation in a 10 mW nuclear reactor. The activated sample is retrieved and the following results obtained:

### RESULTS

At Calibration: 82.0 microcuries

Radionuclidic Purity 98.12% of Lu-177, E=208 keV

Neutron Flux Rate: 5×10¹² n/cm².sec.

Position: 19-5X

Irradiation Time: 11 hours

Decay Time Allowed: 48 hours

Uniformity of Radiation Delivery Along the Wire Was Demonstrated By Autoradiography

### EXAMPLE 6

A 0.0058″ (314 mm length) wire sample of the radioactivatable alloy of Example 1 (53.1 weight percent nickel 0.1 weight percent lutetium, and 44.8 weight percent titantium) weighing 33.4 mg.—containing approximately 0.0334 mg. of lutetium—is placed in a quartz glass protected with aluminum foil. The tube is placed into an aluminum capsule holder, pressure sealed using an inert gas and welded shut. The capsule is inserted into a reactor channel position by hydraulic means and activated by neutron activation in a 10 mW nuclear reactor. The activated sample is retrieved and the following results obtained:

### RESULTS

Activity at Calibration: 1,620 microcuries

Radionuclidic Purity 91.68% of Lu-177, E=208 keV

Neutron Flux Rate: 5×10¹³ n/cm².sec.

Position: 1-4-6

Irradiation Time: 6 hours

Decay Time Allowed: 16 hours

Uniformity of Radiation Delivery Along the Wire Was Demonstrated By Autoradiograph

### EXAMPLE 7

A 0.0058″ (365 mm length) wire sample of the alloy of Example 1 (53.1 weight percent nickel 0.1 weight percent lutetium, and 44.8 weight percent titantium) weighing 38.0 mg.—containing approximately <0.038 mg. of lutetium—is placed in a quartz glass protected with aluminum foil. The tube is placed into an aluminum holder, pressure sealed using an inert gas and welded shut, and inserted into a reactor channel by hydraulic means and activated by neutron activation in a 10 mW nuclear reactor. The activated sample is retrieved and the following results obtained:

### RESULTS

Activity at Calibration: 809 microcuries

Radionuclidic Purity 93.07% of Lu-177, E=208 keV

Neutron Flux Rate: 2.63×10¹³ n/cm².sec.

Position: B3-8Y

Irradiation Time: 9.5 hours

Decay Time Allowed: 48 hours

The foregoing data confirms the attainment of the activity (10, 20, 50, 100 microcuries) or even greater in reactor position of higher flux rates within the RP10 or other nuclear reactor. The radiation isodose, determined by autoradiography, is deemed to be uniform along the length of the activated NiTiLu wire samples as a result of irradiation by neutron activation.

While the invention has been described in connection with exemplary embodiments thereof, it will be understood that many modifications will be apparent to those of ordinary skill in the art, and that this application is intended to cover any adaptation thereof. Therefore, it is manifestly intended that this invention be only limited by the claims and the equivalents thereof.

I claim:

1. A radioactivatable composition for forming a medical device comprised of a biodegradeable polymer containing in uniform dispersion of from 0.05 to about 10.00 percent by weight of a radioactivatable isotope having a half life, when activated, of less than two months.

2. The radioactivatable composition for forming a medical device as defined in claim 1 wherein said radioactivatable isotopes is selected from the group consisting essentially of lutetium-177, samarium-153, cerium-137, 141 and 143, terbium-161, holmium-166, erbium-166 and 172, thulium-172, ytterbium-169, actinium-225, astatine-211, cerium-137, dysprosium-165, erbium-169, gadolinium-148, 159, holmium-166, iodine-124, titanium-45, rhodium-105, palladium-103, rhenium-186, 188, scandium-47, samarium-153, strontium-89, thulium-172, vanadium-48, ytterbium-169, yttrium-90, silver-111, and mixtures thereof.

3. The radioactivatable composition for forming medical devices as defined in claim 1 wherein said radioactivatable isotope is principally a beta particle emitter and having a half-life of at least 24 hours and less than about 60 days.

4. The radioactivatable composition for forming medical devices as defined in claim 1 wherein said biodegradeable polymer is selected from the group consisting of lactides, glycosides, caprolactones, oxyalkenes, polyurethane, polyamides, polyvinylchloride, methy/methylacrylate and mixtures thereof.

5. The radioactivatable composition for forming medical devices as defined in claim 4 including graft and copolymers thereof.

6. The radioactivatable composition for forming medical devices as defined in claim 1 wherein said radioactivatable composition being radioactivatable by being subject to radiation level of from 20 microcuries to 50 milicuries.

7. The radioactivatable composition for forming medical devices as defined in claim 2 wherein said radioactivatable isotope is lutetium-177.

8. The radioactivatable composition for forming medical devices as defined in claim 7 wherein said radioactivatable composition after activation has a storage period up to about 20 days.

9. A stent formed from the radioactivatable composition as defined in claim 1 and further coated with a hydrogel.

10. The stent defined in claim 9 wherein said hydrogel includes a therapeutic drug.

11. The stent formed from the radioactivatable composition defined in claim 10 and further including an anti-thrombotic compound.
